(19) United States
(12) Patent Application Publication
(10) Pub. No.: US 2011/0118537 A1
(43) Pub. Date: May 19, 2011

(54) METHODS AND DEVICES FOR TREATING HEART FAILURE

(76) Inventor: Richard Wampler, Loomis, CA (US)

(21) Appl. No.: 12/939,820

(22) Filed: Nov. 4, 2010

Related U.S. Application Data
(60) Provisional application No. 61/258,122, filed on Nov. 4, 2009.

(51) Int. Cl. A61M 1/12

(52) U.S. Cl. 600/17; 600/16

(57) ABSTRACT

Systems and methods for delivering a miniaturized blood pump configured to draw partially desaturated blood via the femoral vein from the inferior or superior vena cava. A cannula connected to the pump exits the femoral vein and is connected to the femoral artery with a cannula or vascular graft. The pump receives power from a percutaneous lead which runs parallel to the flexible cannula and then exits via a percutaneous opening in the skin. The pump in the venous system removes venous blood and pumps it into the femoral artery. In so doing pressure in the aorta is increased and back pressure in the venous system is decreased.

### CROSS-REFERENCE TO RELATED APPLICATIONS

This application claims priority from U.S. provisional application Ser. No. 61/258,122 filed on Nov. 4, 2009, herein incorporated by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to methods and devices for treating heart disease, and more particularly to methods and devices for assisting the circulation of a failing heart.

2. Description of Related Art

Congestive heart failure (CHF) is a major global public health problem that results in hundreds of thousands of deaths and incalculable human suffering in millions of people each year. Congestive heart failure is a condition in which the heart is unable to adequately pump blood throughout the body due to weak heart muscle contractility. As a result the heart dilates and blood backs up into the lungs, compromising gas exchange from pulmonary edema. Congestive heart failure is a disabling, progressive often fatal disease with no known cure.

First line treatments include modern pharmacologic agents such as ACE inhibitors, beta blockers and diuretics and cardiac resynchronization therapy with a duel chamber pacemaker. When patients become refractory to these first line therapies their best hope for extended survival and improvement in life quality is cardiac transplantation. Unfortunately, there are only approximately 2,000 donor hearts each year for an estimated 75,000 patients who could benefit from cardiac transplantation. Mechanical circulatory assist devices (MCADs) have been developed as a potential alternative to cardiac transplantation.

Mechanical circulatory assist devices are based on blood pumps that function to pump all or part of the cardiac output to relieve the heart of work and to increase peripheral perfusion. The most commonly used MCADs are left ventricular assist devices (LVADs), which unburden the left ventricle. Left ventricular assist devices remove oxygenated blood from the left ventricle or left atrium and pump it into the systemic circulation via the aorta or a peripheral vessel. These devices require major surgery with general anesthesia, cardiopulmonary bypass and are performed by cardiac surgeons. A number of LVADs based on rotary technology or positive displacement technology are now commercially available and are used, on a limited basis, to treat late stage heart failure.

Left ventricular assist devices are most commonly used as a bridge to cardiac transplantation and, on a limited basis, for the palliation of severe heart failure patients who could benefit from cardiac transplantation but for whom a donor heart is not available, i.e. destination therapy

Although CHF was previously believed to be irreversible, significant spontaneous recovery in left ventricular function has been observed in some bridge patients awaiting donor hearts. In many of those patients who experienced spontaneous recovery of left ventricular function, it has been possible to remove the assist device and delay or avoid the need for cardiac transplantation.

If significant left ventricular recovery can occur in patients with very advanced heart failure, the use of mechanical circulatory assistance in patients with less advanced disease i.e., class III b and IV a, may arrest or reverse the fundamental pathology of CHF in large numbers of patients. If this were true, LVADs could offer another alternative for treatment of CHF.

Intravascular transvalvular ventricular assistance taught by Wampler (U.S. Pat. Nos. 4,625,712 & 4,817,586) demonstrated significant clinical benefits in the setting of acute cardiogenic shock, failure to wean from cardiopulmonary bypass, assisted high risk angioplasty and, beating heart coronary revascularization. This device, the Hemopump™, was based on a miniaturized axial flow blood pump which could be inserted via the femoral artery.

Another concept presently under development is transeptal access of blood from the left atrium that is then directed to a rotary pump which directs blood into the systemic circulation. Transeptal access of the left atrium is technically difficult to achieve, particularly from a superior approach such as the subclavian vein. In addition, it is not a popular technique and the procedure is limited to a small number of cardiologists in tertiary centers. The fact that most cardiologists are not accomplished in this method would be a significant barrier to acceptance by clinicians and market penetration.

If the need for accessing fully oxygenated blood from the left atrium or left ventricle could be removed, introduction of mechanical circulatory assistance could be vastly simplified and adopted by cardiologists not accomplished in transeptal left atrial access. The need for a cardiovascular surgeon for accessing the left atrium or left ventricle could also be eliminated. A method of veno-arterial pumping would make it possible to achieve these objectives.

Accordingly, an objective of the present invention is to shift the primary goal of the treatment of CHF from the palliative treatment of symptoms to the treatment of the underlying progressive pathology in order to reverse the primary ventricular pathology. Another objective is the use of mechanical circulatory assistance as a therapeutic modality rather than as a bridge to cardiac transplantation and palliation for end stage patients. A further objective is a mechanical circulatory assistance device (MCAD) that may be implanted via a minimally invasive procedure, and particularly, without requiring a cardiac surgeon or cardiopulmonary bypass for placement. Another object is an MCAD which could be implemented by a cardiologist in the cardiac catheterization laboratory.

The various aspects, modes, embodiments, and features of the present invention, as herein described, variously address certain existing needs such as just described, as well as others, in addition to overcoming and improving upon other shortcomings and deficiencies observed in prior efforts and previously disclosed devices.

### BRIEF SUMMARY OF THE INVENTION

The present invention includes minimally invasive methods and devices for implementing chronic veno-arterial pumping of partially desaturated venous blood into the systemic circulation in patients.

The present invention provides methods and devices for minimally and less invasive implantation of mechanical circulatory assist devices to affect veno-arterial pumping. The methods and devices of the present invention are particularly useful treatments of congestive heart failure, as they can be inserted with minimally or less invasive techniques and can be used as an ambulatory chronic mechanical circulatory assist device to treat patients with CHF, and more particularly therapeutic mechanical circulatory assistance available to class III as well as class IVa congestive heart failure patients. The present invention could be inserted by a cardiologist alone or in tandem with a peripheral vascular surgeon, and would lower the risk of mechanical circulatory assistance for the treatment of congestive heart failure, without the need for cardiac surgical support and without the need for a thoracotomy.

In a preferred embodiment, the device can be inserted in much the same fashion as the implantable defibrillator, while in certain circumstances perhaps to be supplemented with the aid of a vascular surgeon.

One aspect of the present invention provides a device comprising a miniaturized blood pump for placement via the femoral vein into the inferior or superior vena cava. A cannula connected to the outflow of the pump exits the femoral vein and is connected to the femoral artery with a cannula or vascular graft. The pump receives power from a percutaneous lead which runs parallel to the flexible cannula and then exits via a percutaneous opening in the skin. The pump in the venous system removes venous blood and pumps it into the femoral artery. In so doing pressure in the aorta is increased and back pressure in the venous system is decreased. Power is provided to the pump by a percutaneous lead which is connected to an externally worn motor controller and rechargeable battery pack.

One aspect of the present invention accordingly provides a device comprising a cannula for placement in a femoral vein and a cannula for placement in a femoral artery. The venous cannula has continuity with the inlet of a subcutaneously implanted blood pump and the arterial cannula is connected to the outlet of the same pump. Power is provided to the pump via a percutaneous lead which connects to externally worn controller and rechargeable batteries. In this fashion venous blood can then be pumped into the arterial circulation.

In a mode of this aspect, a collapsible thin walled tube can be placed in the femoral vein such that access to the vein is established and semi-rigid walls deployed to maintain patency of the vein lumen and to prevent collapse of the venous wall.

In another aspect of the invention, vascular access to the femoral vein and artery can be established with surgical anastomosis of vascular grafts to the femoral vein and artery. Interposed between the grafts is a subcutaneously implanted blood pump which moves venous blood to the arterial side of the circulation.

In a mode of this aspect, re-enforcement of the venous graft is provided to prevent collapse of the graft walls from negative pressure.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 illustrates a schematic diagram of a veno-arterial pumping system incorporating a venous pump installed within a patient in accordance with the present invention.

FIG. 2 illustrates a schematic diagram of a veno-arterial pumping system incorporating a subcutaneous pump installed within a patient in accordance with the present invention.

FIG. 3 illustrates another schematic diagram of a veno-arterial pumping system of FIG. 1.

FIG. 4 illustrates another schematic diagram of a veno-arterial pumping system of FIG. 2.

FIG. 5 illustrates a cross-sectional view of an inflow cannula of the system of FIG. 1.

FIG. 6 illustrates a cross-sectional view of an alternative inflow cannula of the system of FIG. 1.

FIG. 7 illustrates a cross-sectional view of another alternative inflow cannula of the system of FIG. 1.

FIG. 8 illustrates a cross-sectional view of a collapsible cannula in accordance with the present invention.

FIG. 9 illustrates a cannula coupled to an internal lumen via a vascular graft anastomosis in accordance with the present invention.

### DETAILED DESCRIPTION OF THE INVENTION

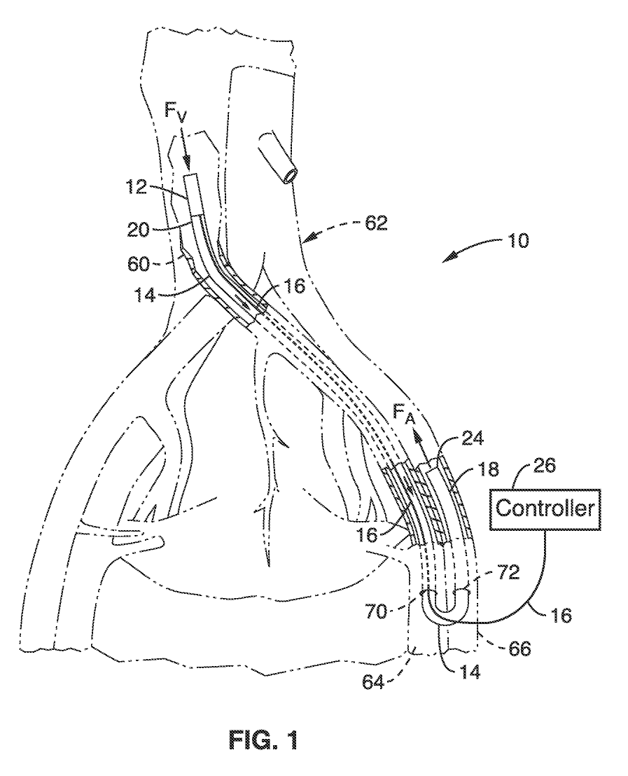

Referring more specifically to the drawings, for illustrative purposes the present invention is embodied in the apparatus generally shown in FIG. 1 through FIG. 9. It will be appreciated that the apparatus may vary as to configuration and as to details of the parts, and that the method may vary as to the specific steps and sequence, without departing from the basic concepts as disclosed herein.

FIG. 1 illustrates a schematic diagram of a veno-arterial pumping system 10 of the present invention. The veno-arterial pumping system 10 comprises a mechanical circulatory support device configured to pump venous blood into the femoral artery without an oxygenator. Partially desaturated venous blood is removed from the venous system and introduced into the arterial circulation. There are three immediate hemodynamic benefits from this method: 1), perfusion pressure, particularly to the heart is increased, 2) part of the work load of the heart is significantly decreased due to volume unloading and 3) the backpressure in the venous system caused by congestive heart failure is significantly reduced.

On initial consideration the value of pumping venous blood into the arterial circulation might seem counterintuitive since venous blood is not fully saturated. However, venous blood is not completely desaturated, but, rather, has an oxygen saturation of about 80%. It has been show in animals and in patients that if the bypass flow of venous blood into the systemic circulation is limited to about ⅓ of the normal cardiac output, oxygen saturations in the thoracic aorta will be at an acceptable level.

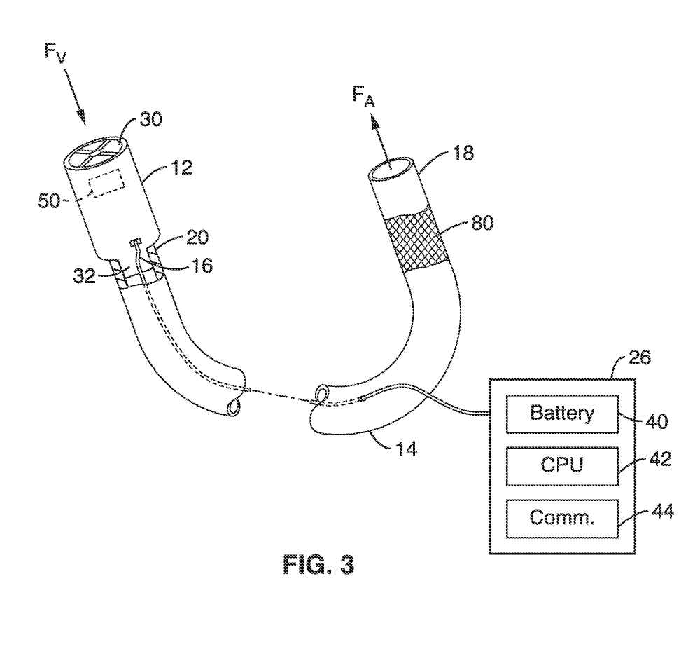

FIG. 1 shows a device 10 for chronic veno-arterial pumping an installed configuration in a patient's body, wherein a small pump 12 is placed in the vena cava 60. The chronic veno-arterial pumping device 10 is shown in an uninstalled configuration in FIG. 3. A distal end 20 of a flexible cannula 14 is connected to the outlet 32 of the intravascular pump 12. The cannula 14 is configured to have a length sufficient to extend from the vena cava 60, upstream along the venous pathway (abdominal vena cava and common iliac vein) to exit out the femoral vein 64 at location 70, and then enter the femoral artery 66 at location 72 such that proximal end 18 extends upstream into the femoral artery 66.

The pump 12 preferably comprises an axial pump (preferably 4-10 mm in diameter) sized to be positioned into the vena cava 60 via the femoral vein 64. Such a small diameter pump would be readily achieved with an axial flow or mixed flow hydraulic design, as shown and described in U.S. patent application Ser. No. 12/324,430, filed on Nov. 26, 2008, herein incorporated by reference in its entirety. The pump 12 comprises an inlet 30, which may be an axial inlet as shown in FIG. 3, or one or more radial side holes (not shown) that is configured to draw venous blood flow F_{V} into the pump and out exit 32 into cannula 14. The venous blood is drawn through the cannula 14 out distal opening 24 of the cannula into the arterial flow F_{A} of the femoral artery 66.

Power to the pump 12 and control of the pump is provided by lead bundle 16, which extends from the pump 12 along the cannula 14 out the femoral vein 64. Lead 16 may comprise a plurality of wires that provide power and/or control signals to the pump 12. The lead 16 then exits the skin and is connected to an externally worn motor controller 26. Controller 26 preferably comprises logic/CPU 42 for sending control signals to the pump 12 via lead bundle 16, and a rechargeable battery 40 for providing power to the motor. The controller 26 may optionally comprise a communication means 44 for sending or receiving data or signals to an external device (not shown).

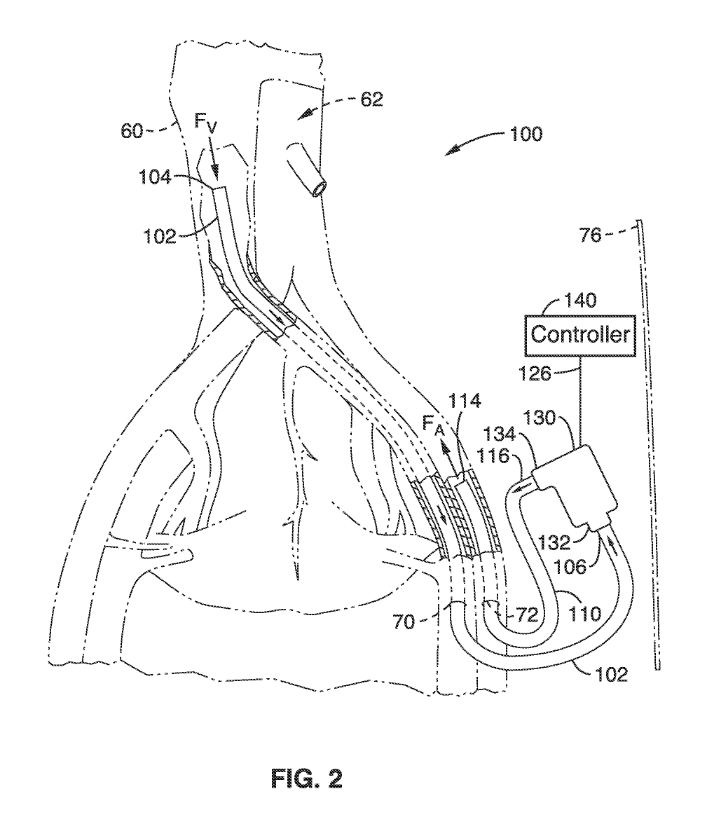

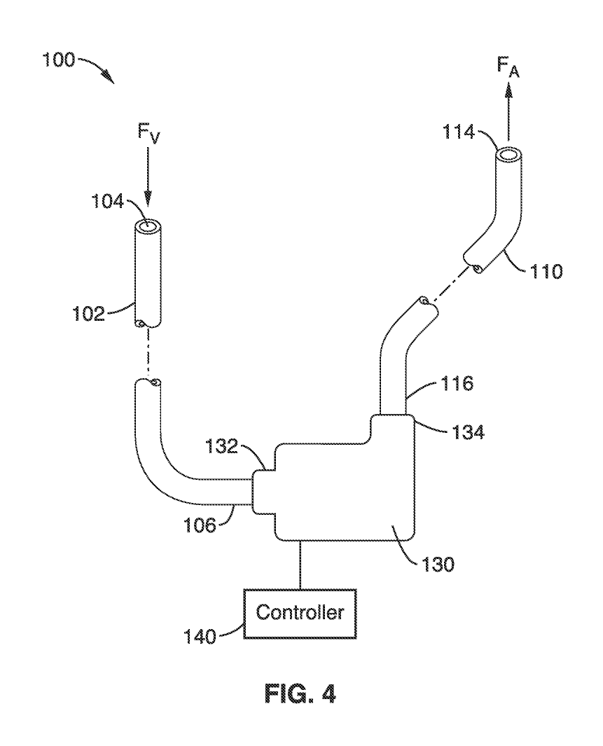

FIG. 2 shows an alternative embodiment of a device 100 for chronic veno-arterial pumping. Device 100 comprises venous and arterial intravascular cannulae, which are configured to be positioned in the femoral vein and artery, respectively, and coupled to miniaturized pump 130 that is implanted subcutaneously in the abdominal wall. The venous inflow cannula 102 is configured to be advanced into the femoral vein 64 at location or aperture 70, such that the distal end 104 extends up the femoral vein 64, common iliac vein, abdominal vena cava and into the superior vena cava 60. The proximal end 106 of cannula 102 extends out from the femoral vein perforation 70 to couple to the inlet 132 of subcutaneously implanted blood pump 130, as shown in greater detail in FIG. 4. An arterial cannula 110 is connected to the outlet 134 at the proximal end 116 of the cannula 110, and the distal end 114 is configured to be inserted through perforation 72 in femoral artery and advanced into the artery.

While the femoral artery 66 is shown as the vessel for directing the venous blood, it is appreciated that any systemic arterial vessel may be chosen. For example, the cannula 110 (or proximal end of cannula 14 in FIG. 1) may be directed into the iliac artery, or anywhere upstream or downstream from location 72 illustrated in FIGS. 1 and 2. Thus, the entry location 72 for cannula 110 (or 14) may be located from among any systemic artery, or be fed within the systemic arterial circulation such that distal end 114 (or 18) is located from among a plurality of locations. Correspondingly, entry location 70 for the intake cannula 102 (or 14) may be at the femoral vein 64, or any other vein in the systemic venous circulation. The distal end 104 of intake cannula 102 (or 14) may also be advanced to an intake location upstream or downstream of vena cava 60.

It is also appreciated that cannula 110 could also be a vascular graft surgically anastomosed to the femoral or iliac artery. For example, graft 150 may be directly connected to outlet 134 of pump 130.

When the pump 130 is operated, blood F, from the vena cava 60 is drawn into distal opening 104 and advanced down cannula 102 to pump 130, where it is the force though outlet 134 and into the arterial cannula 110. The venous blood is then advanced into the femoral artery flow F_{A}.

In the embodiment shown in FIG. 2, the pump 130 is via lead 126 coupled to controller 140 implanted below the skin 76. The controller 126 may be configured to communicate transcutaneously through the skin 76 with an external device via a communication module 44 and CPU 42 as shown in FIG. 1 (e.g. the communication module 44 may comprise an IR transceiver or the like for wireless transmission). In addition, the battery 40 may be charged via induction from an external device.

Alternatively, a percutaneous wire, such as lead 16 shown in FIG. 1, provides power and control to the pump 130 via an externally worn controller and rechargeable battery pack.

Thus, in the embodiments 10, 100 shown in FIGS. 1 and 2, partially desaturated blood F_{V} from the vena cava 60 of the venous system is pumped into to the systemic arterial circulation F_{A} at the femoral artery 66. Generally, the larger the volume of venous blood pumped into the arterial system, the greater the effect of decompression of the venous circulation and, correspondingly the greater the degree of decompression of the left ventricle. The synergy of decompression of the left ventricle and venous circulation in concert with increasing the perfusion pressure of the heart sets the stage for reversal of the primary ventricular pathology. However, too large of a volume of desaturated blood bypassed into the arterial circulation may also lead to undesirable side effects (e.g. the patient may become hypoxic or experience claudication of the lower extremity). Generally, arterial saturation Sₐ0₂ blood to the heart and brain should be at approximately 93%-98%, and not below 90%. Thus, it is desirable to balance the bypassed flow rate to an ideal volume/flow rate that maximizes the benefit of the flow diversion without unduly compromising the oxygen saturation of the arterial circulation at the level of the renal arteries and above.

It has been found that up to one-third of the total blood flow volume may be bypassed without detrimental effect to the heart, brain and kidneys due to decreased arterial saturation. Accordingly, the pumps 12, 130 are ideally configured to pump at a specified flow rate, nominally 3-5 lpm, to achieve the ideal flow rate for the patient. However, the amount of bypassed blood-flow that each patient tolerates may vary dramatically from patient to patient, and depending on whether the patient is active (e.g. exercise tends to increase flow rate (pulse)) or inactive. In addition, the percentage of diversion that each patient can handle may also vary (e.g. some patients may have better results at a flow rate diversion percentage above or slightly above 33%, while others may benefit from a flow rate diversion percentage below or slightly below 33%).

Thus, the methods and systems of the present invention desirably determine the patients natural or baseline blood flow-rate and adapt the output of the pumps 12, 130 accordingly. The patient's baseline blood flow-rate may be determined by preoperative testing, or by adjusting the flow of the pumps 12, 130 post operatively based on various physiologic measurements. For example, the pumps 12, 130 may comprise variable-speed pumps that are remotely controllable via controllers 26, 140. Thus, a physiologic characteristic (such as arterial oxygen saturation) may be measured simultaneously (e.g. via a pulse oximeter (not shown)) while the flow rate of the pump 12, 130 is incrementally increased to determine the patient's tolerance to the bypassed flow. The maximum or ideal flow rate may then be recorded when the arterial saturation is at its lowest acceptable level. The pump 12, 130 flow rate (or pump setting (e.g. supplied power) corresponding to the flow rate) may then be set at that level, e.g. by storing the setting in memory within logic 42 of controller 26.

Alternatively, the pump 12, 130 may comprise one or more sensors 50 (FIG. 3) that measure a physiologic characteristic of the patient to adjust the pump flow real-time. For example, the sensor 50 may comprise one or more of: a pulse oximeter to measure blood saturation, a flow sensor to measure flow rate, pressure sensor to measure venous backpressure, arterial pressure, or the like. The sensor measurements may be transmitted to the controller 26 via lead bundle 16, wherein the logic/CPU42 processes the signal to determine the speed/output of the pump 12, 130. For example, the sensor 50 may comprise a pulse oximeter integrated with or coupled to pump 12 to measure oxygen saturation (a sensor located at the pump 12 as shown in FIG. 3 could measure venous saturation (SᵥO₂), and/or a sensor coupled to the proximal ends 18, 114 of cannulas 14, 110 respectively would measure arterial saturation (SₐO₂). If arterial saturation falls below a minimum threshold value (e.g. SₐO₂<92% or SᵥO₂<60%) or above a maximum threshold (i.e. high saturation level indicating that the patient may have more tolerance to additional flow diversion), than the controller 26 can vary the pump output under constant feedback until an acceptable threshold is achieved. Thus, the pump 12, 130 will continuously operate under substantially ideal and customized flow, regardless of the activity of the patient.

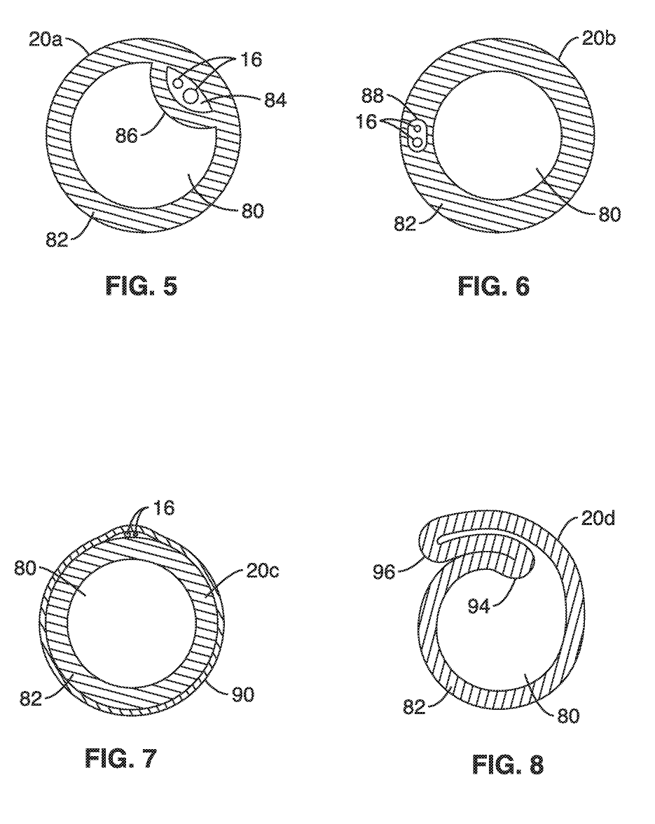

Referring to FIGS. 5-7, the cannulae shown in FIGS. 1-4, and particularly cannula 14 shown in FIGS. 1-3, may be specifically configured to house lead lines 16 at least along a portion of the length of the cannula 14. As shown in the cross-sectional view of FIG. 5, the cannula 14 may comprise section 20a with a thin wall 82 having multiple lumens or channels: a primary internal lumen 80 for transporting blood, and a smaller internal channel 84 separated from flow channel 80 by thin wall 86. Lumen 84 is configured to house lead lines 16 down at least a portion of the length of the cannula 14.

In the cross-sectional view of FIG. 6, the cannula 14 may comprise section 20b comprising a thin wall 82 having a primary internal lumen 80 for transporting blood, and a bore 88 running axially down the length of thin wall 82. Bore 88 is configured to house lead lines 16 down at least a portion of the length of the cannula 14.

In the cross-sectional view of FIG. 7, the cannula 14 may comprise section 20 having a thin wall 82 with an internal lumen/bore 80 for transporting blood. A thin sheath 90, such as shrink-tubing or the like, may be used to restrain lead lines 16 to the outer surface of the thin wall 82 down at least a portion of the length of the cannula 14. It is also appreciated that lead 16 (or lead package comprising a series of individual lead wires) may also be embedded in thin wall section 82 during fabrication of the cannula 14.

The systems 10, 100 have particular performance and design specifications that are unique to the minimally invasive approach disclosed herein. Blood pumps 12, 130 preferably are capable of delivering from 3 to 5 lpm of flow at 120 mm Hg pressure and able to pump for up to 10 years without significant wear or thrombus formation. Total power requirements should be, nominally, 5 watts, with minimal heat dissipation into the body. All materials are preferably biologically compatible and resistant to thrombosis

Subcutaneous pumps, 130 are preferably small enough in external dimension to minimize the size of the implant pocket and produce minimal cosmetic impact or significant pressure on adjacent tissue. A thickness of diameter of no more than 2.0 cm and a greatest dimension of no more than 6 cm is desirable.

The intravascular pump 12 shown in FIG. 1 is ideally no greater than 10.0 mm in diameter and approximately 2-5 cm in length to minimize obstruction of blood flow.

Owing to the anatomical limitations of the peripheral vessels (e.g. femoral vein 64 and femoral artery 66), it is desirable to minimize the outer diameters of the cannulae (14, 102, and 110) and intravascular pumps 12. Cannulas 14, 110 and pumps for venous placement are ideally no larger than 10 mm in diameter. Arterial cannulae 110 should be less than about 6 mm in diameter.

Referring to the cross-sectional view of FIG. 8, the cannulae 14, 102, 100 may be collapsible to form a smaller profile 82 while being delivered to the desired locations within the lumens 64, 66. As shown in FIG. 8, wall 82 may be collapsed into one or more folds 94, 96 to decrease the overall profile during transport, and then expanded when the target location for the cannula is reached.

Cannulae 14, 102, and 110 are preferably thin-walled, reinforced and made of flexible or elastomeric materials with thromboresistant properties. The polymers used in the distal expandable region can include materials such as, but not limited to, polyethylene, HDPE, LDPE, polyethylene blends, Hytrel, Pebax, and the like.

As shown in FIG. 3, cannulae 14, 102, and 110 may all include malleable reinforcing structures 80, and particularly cannula 14 to maintain the sheath in its second, larger, cross-sectional configuration. The reinforcing elements 80 can comprise structures such as, but not limited to, spiral windings of flat or round wire, braided elements of polymeric strands, wire, a mesh structure similar to a stent, a slotted tube with overlapping longitudinally oriented slots, or the like.

Malleable materials such as the polyethylene materials plastically deform under force and offer the benefit of remodeling from a small diameter flexible structure to a large diameter.

In yet other embodiments, the reinforcing structures 80 can comprise shape-memory reinforcing elements that can be heated or cooled to generate austenite or martensite conditions, respectively, that further can be used to drive the cannulae 14 wall 82 from one cross-sectional configuration to another.

In one embodiment, cannulae 14 may comprise an inner layer (not shown) fabricated from lubricious materials such as, but not limited to, polyethylene, HDPE, LDPE, blends of HDPE and LDPE, PTFE, FEP, PFA, Hytrel, Pebax, or the like. Reinforcing structures 80 may then comprise mesh layers applied over the inner layer and in between an outer layer of polymeric material.

The mesh 80 can be formed from a braid, weave, knit or other structure formed into a tubular cross-section. The mesh 80 can be fabricated from polymers such as, but not limited to, polyethylene naphthalate (PEN), PET, polyamide, polyimide, or the like. The mesh 80 can also be fabricated from metals such as, but not limited to, malleable stainless steel, spring stainless steel, nitinol, titanium, cobalt nickel alloy, tantalum, gold, platinum, platinum alloy, and the like.

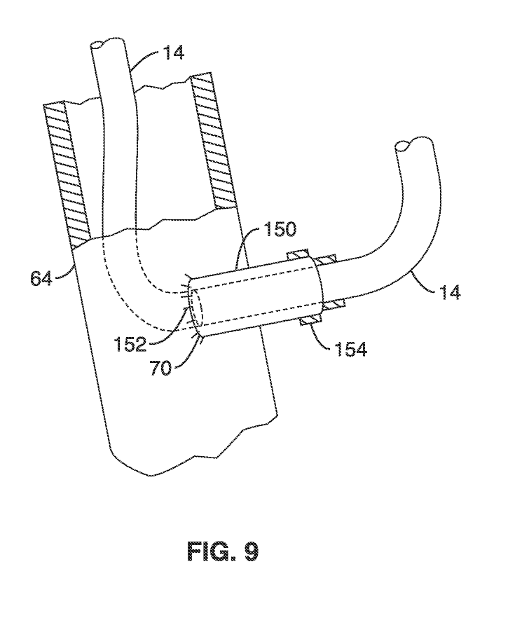

Referring to FIG. 9, outflow cannulae 14, 110 may be coupled to the femoral vein 64 via a vascular graft 150 anastomosed (e.g. end-to-side anastomosis) to the femoral vein 64 at location 70 via stitching 152, staples or like attachment method. A compression band, tie, collar or clamp 154 may be used to secure the graft around the cannulae 14, 110.

Similarly, inflow cannulae 14, 102 may be coupled to the femoral artery 66 with a vascular graft 150. In this configuration, the inflow cannulae 14, 102 may simply only extend to the junction of the graft 150 and the artery 66 wall. Alternatively, the cannulae 14, 102 may extend into the femoral artery a small distance (2-3 inches) as shown in FIGS. 1 and 3. Vascular grafts 150 can be of commonly available commercial types, but should be externally reinforced to prevent kinking.

The systems 10, 100 are configured to be installed in a minimally-invasive process based on transvascular techniques (e.g. Seldinger technique) familiar to the interventional cardiologist. First, a needle, trocar or the like may be inserted into the body below the inguinal ligament and just medial to the location 70 of the femoral vein. If a vascular graft 150 is to be placed, it is anastomosed to the femoral vein (and/or femoral artery). A Seldinger guide wire (not shown) may be directed to into the femoral vein and delivered to the target location within the vena cava 60. The inflow cannula 14, 102 may be guided to the vena cava 60 over the guide wire (e.g. with fluoroscopic guidance).

For the system 10 of FIG. 1, the proximal end 24 of cannula 14 is inserted into perforation 72 of the femoral artery (or attached to arterial graft 150). For system 100 of FIG. 2, the distal end 106 of inflow cannula 102 is attached to input 132 to pump 130, and the outflow cannula 110 attached to the outflow 134 of pump 130 is then fed into femoral artery 66 at location 72 (or attached to arterial graft 150). The pump 130 is positioned to a subcutaneous location within the abdominal wall. In both systems 10, 100, the lead lines 16 and 126 are fed out percutaneously out of the skin to connect to external controller 26.

It is to be appreciated that significantly beneficial objectives of minimally invasive and less invasive insertion methods are permitted by the systems 10, 110 of the present invention, as herein described herein and apparent to one of ordinary skill. The following particular methods for less invasive surgical implantation are envisioned, limitation, to include: 1) insertion without vascular anastomosis, and 2) insertion with vascular anastomosis, 3) insertion of a miniature pump in the venous system (10) and 4) placement of a pump (100) in the subcutaneous tissue of the abdominal wall.

Minimally invasive implementation of the systems of the present invention is considered of particular benefit to the extent that it allows the implementation of mechanical circulatory assistance without a thoracotomy, cardiopulmonary bypass or atrial septal cannulation or touching the heart. Central vascular access is considered of particular benefit to the extent that it is achieved via peripheral vascular access using fluoroscopic guidance for the placement of either an intravascular pump or specialized cannulas.

Minimally invasive placement of the present invention is generally considered to fall, predominately, within the domain of the interventional cardiologist. The methods and devices of the present invention are particularly suited for adaptation for use by such an interventionalist, in particular in that the devices disclosed herein generally allow at least one of, and preferably more than one or all of: 1) a simple means for achieving non-thoracotomy vascular access, 2) small cannula systems and miniature pumps suitable for insertion in peripheral vessels, 3) small pumps suitable for subcutaneous implantation, 4) small pumps suitable for intravascular placement and 5) pumps capable of operating reliably for years in an ambulatory setting. An ability to provide minimally or less invasive implantation of mechanical circulatory assistance capable of operating reliably in extended ambulatory patients is a particular benefit provided by the systems and methods of the present invention.

The pump systems 10, 100, implant configuration, and surgical method shown and described with reference to FIGS. 1 and 2 can be conducted without requiring anastomosis of inflow or outflow cannulas to major vessel walls. It is also to be appreciated that these non-anastomotic methods could be adapted without the need for cardiopulmonary bypass.

It is appreciated that the systems 10, 100 above may be implemented in the femoral artery and vein of either the left or right leg of the patient. However, it is also appreciated that to avoid ischemic conditions in the leg, the distal end 24 or 114 of the outflow cannula 14 or 110 may be elongated to extend upstream of the branches of the femoral arteries (e.g. in the abdominal vena cava. Alternatively, the outflow cannula 14 or 110 may comprise a Y or T junction (not shown) that directs the venous flow to both the left and right common femoral arteries.

FIGS. 1-2 and the disclosure provided above are directed to implantation within human anatomy for treatment of congestive heart failure and associated disease. However, it is appreciated that the various embodiment illustrated above may be also be modified and implemented accordingly for the treatment of animals (e.g. in a canine presenting mitral valve disease or congestive heart failure), or for other cardiovascular disorders that may benefit from such venous to arterial circulation bypass.

While this invention has been described in conjunction with the specific embodiments outlined above, it is evident that many alternatives, modifications and variations will be apparent to those skilled in the art. For example, a number of different pumping technologies could be used to provide venoarterial pumping either of continuous flow and positive displacement designs. Also, the figures depict venous and arterial access being from the same side, but contralateral access would be acceptable. Although not described in detail, there are also a number of additional combinations of vascular accesses possible in which cannulae could be replaced with vascular grafts and vice versa.

From the discussion above it will be appreciated that the invention can be embodied in various ways, including the following:

1. An apparatus for treatment of heart failure in a patient, comprising: a cannula having a proximal end and a distal end; wherein the distal end of the cannula is sized to be received at a first access location within an accessible vein of the patient and advanced upstream along the venous circulatory system to an intake location within the venous circulatory system; wherein the proximal end is configured to be coupled to be in fluid communication at a second access location within an accessible artery of the patient; and a pump disposed at the proximal end of the cannula; the pump comprising in inlet configured to receive venous blood from the intake location, and an outlet coupled to the distal end of the cannula; wherein the pump is configured to draw at least a portion of the venous blood from the intake location into the first cannula and direct said portion of the venous blood into the systemic arterial circulation.

2. An apparatus as recited in embodiment 1: wherein the first access location comprises a location along the femoral vein of the patient; wherein the second access location comprises a location along the femoral artery of the patient; and wherein the intake location comprises a location within the vena cava of the patient.

3. An apparatus as recited in embodiment 1, further comprising: a controller; a lead coupling the controller to the pump; wherein the controller is configured to power the pump from a location outside the venous circulatory system.

4. An apparatus as recited in embodiment 3:

wherein the cannula comprises a central channel for diverting blood flow and a secondary channels for housing the lead at least along a portion of the cannula.

5. An apparatus as recited in embodiment 3: wherein the cannula is collapsible to form a collapsed configuration for delivery to the first or second location, and is expandable to form an expanded configuration.

6. An apparatus as recited in embodiment 3: wherein the cannula comprises a reinforced mesh to retain the cannula in the expanded configuration once expanded.

7. An apparatus as recited in embodiment 2: wherein the cannula is coupled to one or more of the femoral vein or femoral artery via an anastomosed graft.

8. An apparatus as recited in embodiment 3: wherein the pump comprises a variable speed pump; wherein the controller comprises a processor for controlling said variable speed pump; wherein the controller is configured to control the speed of the pump to vary flow rate of venous blood into the systemic arterial circulation.

9. An apparatus as recited in embodiment 8, further comprising: one or more sensors coupled to the controller; wherein the one or more sensors are configured to receive data relating to one or more physiological characteristics of the patient; and wherein the controller is configured to process the data and adjust the flow rate according to said data.

10. An apparatus for treatment of heart failure in a patient, comprising:

an inflow cannula having a proximal end and a distal end; wherein the distal end of the inflow cannula is sized to be received at a first access location within an accessible vein of the patient and advanced upstream along the venous circulatory system to an intake location within the venous circulatory system of the patient; a pump having an input configured to be coupled to the proximal end of the inflow cannula at a location external to the venous circulatory system, the pump further comprising an outlet configured to be coupled in fluid communication at a second access location within an accessible artery of the patient; and wherein the pump is configured to draw at least a portion of venous blood from the venous circulatory system into the inflow cannula and direct said portion of the venous blood into the systemic arterial circulation.

11. An apparatus as recited in embodiment 10: wherein the first access location comprises a location along the femoral vein of the patient; wherein the second access location comprises a location along the femoral artery of the patient; and wherein the intake location comprises a location within the vena cava of the patient.

12. An apparatus as recited in embodiment 11, further comprising: an outflow cannula having a proximal end and a distal end; wherein the proximal end of the outflow cannula is couplet to the outlet of the pump; and wherein the distal end of the cannula is coupled to the femoral artery at said second access location.

13. An apparatus as recited in embodiment 10, further comprising: a controller; a lead coupling the controller to the pump; wherein the controller is configured to power the pump from a location outside the venous circulatory system.

14. An apparatus as recited in embodiment 10: wherein the inflow cannula is collapsible to form a collapsed configuration for delivery to the location within the venous circulatory system, and is expandable to form an expanded configuration.

15. An apparatus as recited in embodiment 14: wherein the inflow cannula comprises a reinforced mesh to retain the cannula in the expanded configuration once expanded.

16. An apparatus as recited in embodiment 12: wherein the outflow cannula is coupled to the femoral artery via an anastomosed graft.

17. An apparatus as recited in embodiment 12: wherein the inflow cannula is coupled to the femoral vein via an anastomosed graft.

18. An apparatus as recited in embodiment 10: wherein the pump comprises a variable speed pump; wherein the controller comprises a processor for controlling said variable speed pump; wherein the controller is configured to control the speed of the pump to vary flow rate of venous blood into the systemic arterial circulation.

19. An apparatus as recited in embodiment 18, further comprising: one or more sensors coupled to the controller; wherein the one or more sensors are configured to receive data relating to one or more physiological characteristics of the patient; and wherein the controller is configured to process the data and adjust the flow rate according to said data.

20. A method for treatment of heart failure in a patient, comprising: receiving a distal end of a first cannula at a first access location within an accessible vein of the patient; advancing the distal end of the first cannula upstream along the venous circulatory system to an intake location within the patient; implanting a pump within the patient; coupling the first cannula to the pump; coupling an output of the pump to a second access location within the systemic arterial circulation of the patient; and operating said pump to draw venous blood from the vena cava into the first cannula and direct said venous blood to a the second location within the systemic arterial circulation.

21. A method as recited in embodiment 20: wherein the first access location comprises a location along the femoral vein of the patient; wherein the second access location comprises a location along the femoral artery of the patient; and wherein the intake location comprises a location within the vena cava of the patient.

22. A method as recited in embodiment 20, further comprising: coupling a controller to the pump via a lead; powering the pump with said controller from a location outside the venous circulatory system.

23. A method as recited in embodiment 20, wherein the first cannula is collapsible to form a collapsed configuration for delivery to the location within the venous circulatory system, and is expandable to form an expanded configuration.

24. A method as recited in embodiment 21: wherein the pump comprises an inlet configured to receive venous blood from the vena cava, and an outlet coupled to the distal end of the cannula; and wherein a proximal end of the first cannula is coupled to be in fluid communication with the femoral artery at the second access location.

25. A method as recited in embodiment 21, further comprising: coupling a proximal end of the first cannula to an inlet of the pump; coupling a proximal end of a second cannula to an outlet of the pump at a location external to the venous circulatory system; coupling a distal end of the second cannula to the second access location along the femoral artery; draw at least a portion of venous blood from the vena cava into the first cannula to the inlet of the pump, and directing the venous blood into the femoral artery via the second cannula.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. An apparatus for treatment of heart failure in a patient, comprising:
a cannula having a proximal end and a distal end;
wherein the distal end of the cannula is sized to be received at a first access location within an accessible vein of the patient and advanced upstream along the venous circulatory system to an intake location within the venous circulatory system;
wherein the proximal end is configured to be coupled to be in fluid communication at a second access location within an accessible artery of the patient; and
a pump disposed at the proximal end of the cannula;
the pump comprising in inlet configured to receive venous blood from the intake location, and an outlet coupled to the distal end of the cannula;
wherein the pump is configured to draw at least a portion of the venous blood from the intake location into the first cannula and direct said portion of the venous blood into the systemic arterial circulation.

2. An apparatus as recited in claim 1:
wherein the first access location comprises a location along the femoral vein of the patient;
wherein the second access location comprises a location along the femoral artery of the patient; and
wherein the intake location comprises a location within the vena cava of the patient.

3. An apparatus as recited in claim 1, further comprising:
a controller;
a lead coupling the controller to the pump;
wherein the controller is configured to power the pump from a location outside the venous circulatory system.

4. An apparatus as recited in claim 3:
wherein the cannula comprises a central channel for diverting blood flow and a secondary channels for housing the lead at least along a portion of the cannula.

5. An apparatus as recited in claim 3:
wherein the cannula is collapsible to form a collapsed configuration for delivery to the first or second location, and is expandable to form an expanded configuration.

6. An apparatus as recited in claim 3:
wherein the cannula comprises a reinforced mesh to retain the cannula in the expanded configuration once expanded.

7. An apparatus as recited in claim 2:
wherein the cannula is coupled to one or more of the femoral vein or femoral artery via an anastomosed graft.

8. An apparatus as recited in claim 3:
wherein the pump comprises a variable speed pump;
wherein the controller comprises a processor for controlling said variable speed pump;
wherein the controller is configured to control the speed of the pump to vary flow rate of venous blood into the systemic arterial circulation.

9. An apparatus as recited in claim 8, further comprising:
one or more sensors coupled to the controller;
wherein the one or more sensors are configured to receive data relating to one or more physiological characteristics of the patient; and
wherein the controller is configured to process the data and adjust the flow rate according to said data.

10. An apparatus for treatment of heart failure in a patient, comprising:
an inflow cannula having a proximal end and a distal end;
wherein the distal end of the inflow cannula is sized to be received at a first access location within an accessible vein of the patient and advanced upstream along the venous circulatory system to an intake location within the venous circulatory system of the patient;
a pump having an input configured to be coupled to the proximal end of the inflow cannula at a location external to the venous circulatory system,
the pump further comprising an outlet configured to be coupled in fluid communication at a second access location within an accessible artery of the patient; and
wherein the pump is configured to draw at least a portion of venous blood from the venous circulatory system into the inflow cannula and direct said portion of the venous blood into the systemic arterial circulation.

11. An apparatus as recited in claim 10:
wherein the first access location comprises a location along the femoral vein of the patient;
wherein the second access location comprises a location along the femoral artery of the patient; and
wherein the intake location comprises a location within the vena cava of the patient.

12. An apparatus as recited in claim 11, further comprising:
an outflow cannula having a proximal end and a distal end;
wherein the proximal end of the outflow cannula is couplet to the outlet of the pump; and
wherein the distal end of the cannula is coupled to the femoral artery at said second access location.

13. An apparatus as recited in claim 10, further comprising:
a controller;
a lead coupling the controller to the pump;
wherein the controller is configured to power the pump from a location outside the venous circulatory system.

14. An apparatus as recited in claim 10:
wherein the inflow cannula is collapsible to form a collapsed configuration for delivery to the location within the venous circulatory system, and is expandable to form an expanded configuration.

15. An apparatus as recited in claim 14:
wherein the inflow cannula comprises a reinforced mesh to retain the cannula in the expanded configuration once expanded.

16. An apparatus as recited in claim 12:
wherein the outflow cannula is coupled to the femoral artery via an anastomosed graft.

17. An apparatus as recited in claim 12:
wherein the inflow cannula is coupled to the femoral vein via an anastomosed graft.

18. An apparatus as recited in claim 10:
wherein the pump comprises a variable speed pump;
wherein the controller comprises a processor for controlling said variable speed pump; and
wherein the controller is configured to control the speed of the pump to vary flow rate of venous blood into the systemic arterial circulation.

19. An apparatus as recited in claim 18, further comprising:
one or more sensors coupled to the controller;
wherein the one or more sensors are configured to receive data relating to one or more physiological characteristics of the patient; and
wherein the controller is configured to process the data and adjust the flow rate according to said data.

20. A method for treatment of heart failure in a patient, comprising:
receiving a distal end of a first cannula at a first access location within an accessible vein of the patient;
advancing the distal end of the first cannula upstream along the venous circulatory system to an intake location within the patient;
implanting a pump within the patient;
coupling the first cannula to the pump;
coupling an output of the pump to a second access location within the systemic arterial circulation of the patient; and
operating said pump to draw venous blood from the vena cava into the first cannula and direct said venous blood to a the second location within the systemic arterial circulation.

21. A method as recited in claim 20:
wherein the first access location comprises a location along the femoral vein of the patient;
wherein the second access location comprises a location along the femoral artery of the patient; and
wherein the intake location comprises a location within the vena cava of the patient.

22. A method as recited in claim 20, further comprising:
coupling a controller to the pump via a lead;
powering the pump with said controller from a location outside the venous circulatory system.

23. A method as recited in claim 20, wherein the first cannula is collapsible to form a collapsed configuration for delivery to the location within the venous circulatory system, and is expandable to form an expanded configuration.

24. A method as recited in claim 21:
wherein the pump comprises an inlet configured to receive venous blood from the vena cava, and an outlet coupled to the distal end of the cannula; and
wherein a proximal end of the first cannula is coupled to be in fluid communication with the femoral artery at the second access location.

25. A method as recited in claim 21, further comprising:
coupling a proximal end of the first cannula to an inlet of the pump;
coupling a proximal end of a second cannula to an outlet of the pump at a location external to the venous circulatory system;
coupling a distal end of the second cannula to the second access location along the femoral artery;
drawing at least a portion of venous blood from the vena cava into the first cannula to the inlet of the pump, and directing the venous blood into the femoral artery via the second cannula.
